(19) United States
(12) Patent Application Publication
(10) Pub. No.: US 2015/0147298 A1
(43) Pub. Date: May 28, 2015

(54) Sleep enhancement with cortisol reduction medical food

(71) Applicants: John Edgar Menear, Santa Cruz, CA (US); Joseph Ramaekers, Aptos, CA (US)

(72) Inventors: John Edgar Menear, Santa Cruz, CA (US); Joseph Ramaekers, Aptos, CA (US)

(73) Assignee: CortControl, Inc., Santa Cruz, CA (US)

(21) Appl. No.: 14/544,274

(22) Filed: Dec. 17, 2014

Related U.S. Application Data
(63) Continuation-in-part of application No. 13/843,581, filed on Mar. 15, 2013.
Application No. 13/843,581 is a continuation-in-part of application No. 13/718,319, filed on Dec. 18, 2012.
Application No. 13/718,319 is a continuation-in-part of application No. 12/631,745, filed on Dec. 4, 2009, now abandoned.
Application No. 12/631,745 is a continuation of application No. 11/492,464, filed on Jul. 24, 2006, now abandoned.
(60) Provisional application No. 61/964,100, filed on Dec. 24, 2013.

(51) Int. Cl. A61K 35/74; A23L 1/30; A61K 31/716

(52) U.S. Cl. CPC A61K 35/74; A61K 31/716; A23L 1/3014; A61K 2035/11
USPC 424/93.4

(57) ABSTRACT

A method of using a sleep enhancement medical food to improve human sleep quality. This sleep enhancement medical food consists of transfer factor, lactic acid generating bacteria, and/or glucans in appropriate combinations. The sleep enhancement medical food, administered correctly, reduces cortisol levels. High cortisol causes insomnia. Dosage amounts are adjusted for client weight. Consumption frequency and dosage may be adjusted in response to cortisol measurements. Typically, consumption of the sleep enhancement medical food is done under professional supervision, and may be combined with other treatment strategies.

### CROSS-REFERENCE TO RELATED APPLICATIONS

The current application is a continuation-in-part of non-provisional application U.S. Ser. No. 13/843,581, filed Mar. 15, 2013, which is a continuation-in-part of application Ser. No. 13/718,319, filed on Dec. 18, 2012. application Ser. No. 13/718,319 is a continuation-in-part of application Ser. No. 12/631,745, filed Dec. 4, 2009 and a continuation-in-part of application Ser. No. 11/492,464, filed Jul. 24, 2006. The current application also claims priority to U.S. provisional application 61/964,100 filed Dec. 24, 2013. The above references are incorporated by reference in their entirety into the current application.

### STATEMENT REGARDING FEDERALLY SPONSORED RESEARCH

Not Applicable

### REFERENCE TO A MICROFICHE APPENDIX

Not Applicable

### BACKGROUND OF THE INVENTION

1. Field of the Invention

This invention relates to the treatment of human breast cancer, which is correlated with an excess of cortisol (corticosteroids). More specifically, this invention describes method of feeding a medical food composition, which mitigates breast cancer by reducing a patient's patient's cortisol level. In the United States, 11% of the population reports the inability to get sufficient sleep. That number corresponds to approximately 35 million people.

2. Description of Related Art

Data from (1) the Department of Otolaryngology-Head and Neck Surgery, St. Vincent's Hospital, The Catholic University of Korea, College of Medicine, Suwon, Republic of Korea; (2) Stanford University Sleep Medicine Division, Stanford University, School of Medicine, Stanford, Calif., United States; (3) Department of Psychiatry, St. Vincent's Hospital, The Catholic University of Korea, College of Medicine, Suwon, Republic of Korea; and (4) Suum ENT & Sleep Clinic, Seoul, Republic of Korea showed that salivary cortisol is a useful biomarker for sleep disorder. Eighty (80) people were included into the test, which incorporated controls.

Anecdotal studies also show the connection between cortisol and sleep difficulty. After cortisol reduction, athletes performed better during games and students performed better on tests. In both cases, better sleep the night before was cited as the reason for improved performance.

Cortisol output is initiated when pituitary gland secretions of ACTH stimulate the adrenal glands, which produce the cortisol.

Cortisol is the primary human stress hormone. It is difficult to fall asleep when stressed. Stress (chemically indicated by cortisol) normally causes a surge in adrenal hormones like adrenaline that increase alertness. High cortisol makes it more difficult to relax into sound sleep—especially when levels remain high or rise-and-fall irregularly throughout the night.

Frequent or constant stress can chronically elevate these hormone levels, resulting in a hyper-vigilant state incompatible with restful sleep. If this is the reason for poor sleep, anything that reduces stress and enhances the ability to handle stress may improve sleep. This includes dietary factors.

Normally, cortisol exhibits a periodic rhythm: high in the morning; moderate in the afternoon; and low at night. Both the periodic rhythm and the absolute concentration are consequential to sound sleep. For example, shift work throws off the body's circadian rhythm and may lead to chronic insomnia.

Once diagnosed, the treatment of sleep disorder depends upon the underlying cause. CPAP masks are a common treatment for sleep apnea, but they are not always effective. Further, they are uncomfortable. Over-the-counter sleep aids do not necessarily lead to restful sleep, and a lethargic-style hangover the next day is a common complaint.

Medications for sleeplessness are commonly used. But, if high cortisol is an underlying factor, the effect is muted. A medication might treat symptoms, but leave the underlying issue intact.

High cortisol cases tend to be the most severe sleep disturbances. Highly addictive benzodiazepines (Xanax, Ativan, or Klonopin) are the only class of pharmaceutical in current use capable of temporarily suppressing cortisol levels.

The above treatments are intrusive. A food based approach (alone or in combination with other therapies) is more desirable. The side effects of medical foods are far less than conventional therapies.

A food based solution is particularly appropriate in view of current attitudinal trends.

The consuming public now understands that foods possess more than basic nutrition (protein, carbohydrate, fat, etc.). For example, 95% of consumers agree that foods have health benefits that go beyond basic nutrition and may reduce the risk of disease. More than 50% of U.S. consumers believe that foods can replace the use of drugs.

The Federal Drug Administration acknowledges the trend of augmenting medications with foods. A relatively new category of “Medical Foods” reflects this. Medical foods should be administered or monitored by a doctor, nutritionist, nurse, medical technician or equivalent health care professional. Medical foods are defined by the Federal Drug Administration in 21 CFR 101.9(j)(8).

A sleep enhancement medical food that reduces cortisol is needed as a stand-alone treatment for sleeplessness or as an improvement to conventional remedies.

### BRIEF SUMMARY OF THE INVENTION

Following is a condensed summary of the invention. By necessity, details are omitted in order to simply state the essence of the invention. Omitted details within this section should not be construed in a way that limits the scope of the invention.

The instant invention is a method of treating human insomnia with a medical food that reduces human cortisol levels and simultaneously builds the immune system.

This method is directed to people with sleep disorders where high cortisol is present. If high cortisol is present at any time during sleep, the disclosed treatment method is within the scope of this instant invention. Cortisol levels sometimes cycle between high and low levels during sleeping periods; this still falls within the scope of this instant invention.

This invention does not claim to address all sleep disorders. This invention addresses sleep disorders where high cortisol is a correlated issue.

To be thorough, the inventors note that lower-than-normal cortisol can also disrupt sleep. This is usually associated with low blood sugar, and this low-cortisol scenario is not within the scope of this current invention.

In this disclosure, high cortisol is defined as “above the United States population average” when measured as salivary, serum, hair, or urine cortisol.

A sleep enhancement medical food contains at least transfer factor and lactic acid generating bacteria. Any sleep enhancement medical food treatment that includes consumption of transfer factor and lactic acid generating bacteria is within the scope of this invention. A sleep enhancement treatment that does not include consumption of transfer factor and lactic acid generating bacteria is outside the scope of this invention. Sleep enhancement medical food may include other components in conjunction with transfer factor and lactic acid generating bacteria.

The inventors have found through field trials that feeding a mixture of transfer factor and lactic acid generating bacteria lowers cortisol in mammals. Addition of glucans further enhances the cortisol-lowering effect. The cortisol-reducing property was first determined during controlled cattle studies. After consumption, diseased cattle showed significant health improvement and reduced cortisol, relative to a control group.

During the cattle studies, blood cortisol was measured over time as a chemical marker. As the animals improved, decreased cortisol levels were observed. Cortisol levels in the test cattle (calves) decreased from 34.3 to 21.5 ng/ml.

Although this data involves cattle, cross-over charts indicate that the biochemical response of cattle and humans are similar. Further, transfer factor functions across species. For instance, bovine transfer factor builds immunity on dogs and humans, and ovum transfer factor builds immunity on cattle and humans.

A cortisol-lowering response in humans is observable via dietary inclusion of transfer factor and lactic acid generating bacteria.

For the current method-of-use invention, the central underlying facts are (1) ingesting a food containing transfer factor and lactic acid generating bacteria leads to reduced human cortisol, and (2) high cortisol correlates with sleep difficulty. Lowering cortisol improves sleep because high cortisol is a cause of insomnia.

The immune building action of transfer factor and lactic acid generating bacteria further supports sleep enhancement by lowering physical stresses within the human body.

This application is not limited by any specific mechanism that explains how cortisol is lowered or immune function is increased. Claims are based on observation rather than theory. Claims are based on a medical food formulation that lowers cortisol, increases immune function, and enhances restful sleep—regardless of the mechanism.

Glucans may be added to the combination of transfer factor and lactic acid generating bacteria to increase effectiveness. Other useful additives include gamma amino butyric acid (GABA). GABA is the brain's primary inhibitory (i.e., calming) neurotransmitter. It neutralizes adrenaline as a primary function. Serotonin and melatonin are also appropriate additives in some cases.

There may be many reasons and many factors related to sleep disorders. The instant application method provides a useful remedy where high cortisol is a correlated factor. When high cortisol is a factor, insomnia is particularly problematic. The quality of insomnia is typically an alert “ready to get to work” sensation, or an agitated/hyper-vigilant mindset. In this condition, restful sleep is unlikely.

A human medical food to mitigate the symptoms of cortisol-induced sleep disorder by lowering cortisol levels is described herein. Administration by feeding is discussed in this disclosure. But it would be obvious to a person of ordinary skill that other forms of administration-such as injection, capsule swallowing or intravenous administration-would lead to the same result.

Objects of this invention include some or all of the following: (1) improve sleep quality with a medical food, (2) combine transfer factor and lactic acid generating bacteria to create a medical food, (3) add glucans to the combination of transfer factor and lactic acid generating bacteria, (4) select the amount of each medical food component based on weight, (5) select the dosage weight of each medical food component based on feeding frequency and patient weight, and (6) allow dosage adjustments over time based on cortisol measurements.

### BRIEF DESCRIPTION OF THE SEVERAL VIEWS OF THE DRAWINGS

FIG. 1 shows evening effect of the invented medical food from a cattle study. Note that cortisol levels decreased.

FIG. 2 shows morning effect of the invented medical food from a cattle study. Note that cortisol levels decreased.

### DETAILED DESCRIPTION OF THE INVENTION

A composition of transfer factor and lactic acid generating bacteria was patented by Joseph Ramaekers (a current joint inventor). Refer to U.S. Pat. No. 6,962,718, claim 6, issued Nov. 8, 2005, which recites, “A formulation comprising pharmaceutically acceptable transfer factor and a pharmaceutically acceptable lactic acid generating bacteria wherein the amount of said transfer factor is from 10 mg to 10,000 mg per ounce of formulation”.

This composition has been used successfully as a medical food in the veterinary field for a variety of animal diseases. For example, feedlot cattle showed decreased mortality. Also, mammal fertility was increased.

One of the benefits of this medical food formulation is cortisol reduction. A second benefit is immune system building.

High levels of cortisol reduce REM sleep.

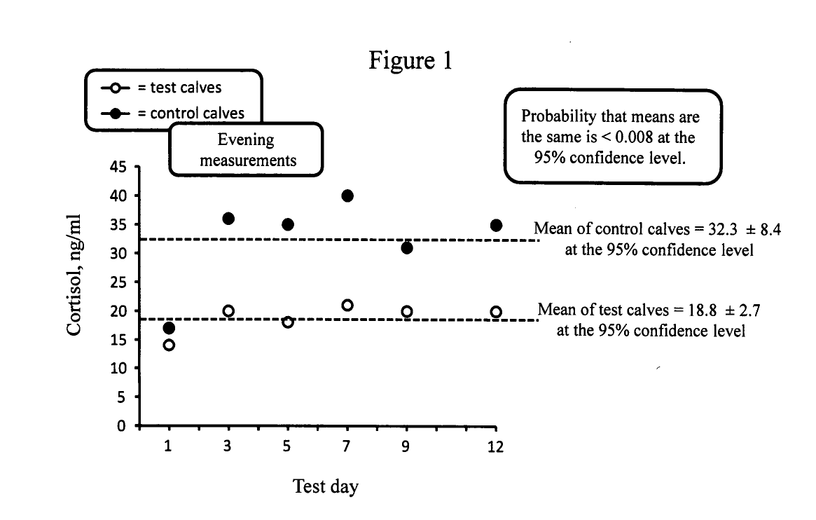

FIG. 1 summarizes the evening cortisol reduction (test versus control) for a calf study over a 12-day period. Our more current research indicates that humans experience a similar evening cortisol reduction after consuming the sleep enhancement medical food.

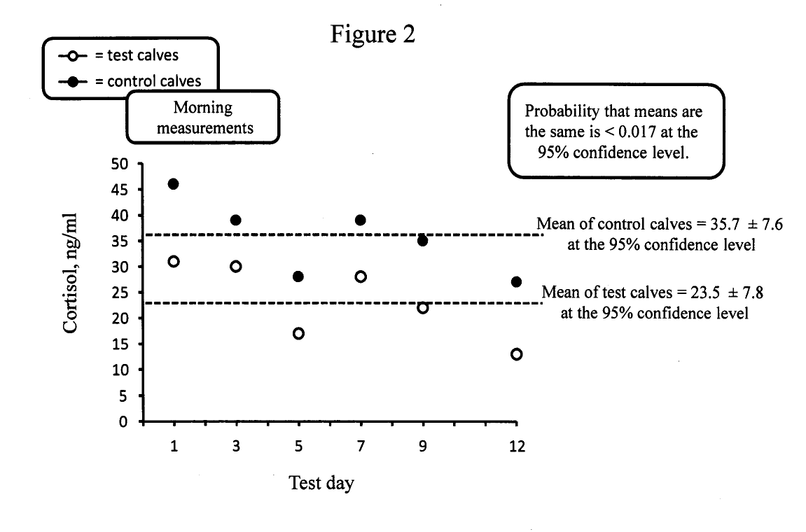

FIG. 2 summarizes the morning cortisol reduction (test versus control) for a calf study over a 12-day period. Our more current research indicates that humans experience a similar morning cortisol reduction after consuming the sleep enhancement medical food.

Laboratory tests have demonstrated the immune building properties of combining transfer factor, lactic acid generating bacteria, and glucans. This medical food combination is capable of increasing the immune system more than five times the baseline level. The metric to demonstrate this five-fold increase was killer T-cell count.

This method-of-use invention is designed for people experiencing sleeping problems, where high cortisol is a correlated factor. An ingested medical food utilizes the human cortisol-reducing and immune-building properties of that medical food to help people with sleep disorders.

Sleep disorders have multiple causes. Not all involve high cortisol. But if a person has both high cortisol and a sleep disorder, that person can be treated via this current invention.

Components of the medical food that improve sleep quality include transfer factor and lactic acid generating bacteria. Glucans may be added to increase effectiveness. Following is a description of each component.

Transfer factor is produced by leucocytes and lymphocytes. Transfer factor comprises small water soluble polypeptides of about 44 amino acids that stimulate or transfer cell mediated immunity from one individual to another.

The properties, characteristics and processes for obtaining transfer factor are discussed in U.S. Pat. Nos. 4,816,563; 5,080,895; 5,840,700, 5,883,224 and 6,468,534, the contents of which are hereby incorporated by reference into the present application.

Alternative sources of transfer factor include avian transfer factor, ova transfer factor, and colostrum from goats, pigs, horses and humans. This listing is not complete. In addition, combinations of transfer factors from any number of sources may be used in human sleep improvement formulations.

In certain embodiments to address sleep disorder, a significant fraction of transfer factor has a molecular weight of less than 10,000 Daltons. Transfer factor may be derived from colostrum, but it is considerably different from colostrum. It takes approximately 1000 grams of colostrum to produce 1 gram of the transfer factor used for sleep improvement.

Transfer factor is commercially available, and known to be safe. Transfer factor is included in the Physician's Desk Reference.

Lactic acid generating bacteria is a necessary component of the sleep improvement medical food, and is GRAS (generally recognized as safe). Lactic acid generating bacteria support digestion and brain health. Lactic acid generating bacteria provide healthful effects that are found in non-pasteurized sauer kraut and cod liver oil. Within the intestinal tract, lactic acid generating bacteria are beneficial. It has been estimated that 80% of human health depends on beneficial intestinal bacteria. Intestinal bacteria account for 90% of the total human cell count.

Lactic acid generating bacteria is only one component of the sleep improvement medical food, but it is an important inclusion.

A human body becomes stressed by poor digestion, and cortisol levels will increase to reflect that stress. Stated differently, lactic acid generating bacteria helps reduce cortisol via improved digestion.

Glucans (polysaccharides) are known to support the immune system. When combined with transfer factor and lactic acid generating bacteria, a synergy is created. The combined effect on sleep disturbance is greater than the effect predicted from summing the individual components. Glucans may be present as natural or hybrid mushrooms.

The method of using transfer factor, lactic acid generating bacteria, and glucans for treating insomnia can also be viewed as two improvements to U.S. Pat. No. 6,962,718. The two improvements (limitations) are (1) the addition of glucans, to the basic medical food and (2) the specific method of using the patented composition to improve sleep quality. Improvement claims are presented in the claims section.

A sleep enhancement medical food may be augmented with additional additives. Example additives are minerals, probiotics, prebiotics, dimethyl glycine, ascorbic acid, Vitamin A, Vitamin B1, Vitamin B2, Vitamin B12, Vitamin D3, Vitamin E, dipotassium phosphate, potassium chloride, magnesium sulfate, calcium pantothenate, minerals, antioxidants, amino acids, nutraceuticals, inositol hexaphosphate (Ip6), mannans, olive leaf extract, and phytosterols. In certain preferred embodiments, mannans are derived from Aloe Vera. In certain preferred embodiments, phytosterols may be derived from soya bean.

Probiotic additives include, but are not limited to B. subtlis, B. longum, B. thermophilium, B. coagulans, E. faecium, S. cerevisia, L. casei, L. plantarum, Pediococccus acidilacticii, Kluyveromyces marxianus fragillis, and combinations thereof.

The above listings do not include all possible additives. The food compositions may also include one or more of the following: carrier proteins such as serum albumin; buffers such as sodium acetate; fillers such as microcrystalline cellulose, lactose, corn and other starches; binding agents; sweeteners and other flavoring agents; coloring agents; and polyethylene glycol. Additives are well known in the art, and are used in a variety of formulations.

Neurotransmitter precursors such as GABA, tryptophan, and/or melatonin may be added to the sleep improvement medical food.

The relative proportion of transfer factor, lactic acid generating bacteria, and glucans within the composition may vary widely. And dosage levels can be adjusted. The reasons for the wide ranges are (1) that humans can create cortisol by imagining threatening scenarios, and (2) that different stages of treatment are involved.

However, some reasonable weight ranges for transfer factor are 0.05-50 mg per pound of body weight. Reasonable weight ranges for lactic acid generating bacteria are 0.47-10 mg per pound of body weight. This is based on a nominal live count of 2.5×10⁶ CFU/Ounce. Reasonable weight ranges for glucans are 0.1-10 mg per pound of body weight.

A method of using sleep enhancement medical food may have some or all of the following steps:
(1) determine that high cortisol and the sleep disorder are both present,
(2) select the correct proportion of transfer factor, lactic acid generating bacteria, and glucans,
(3) choose the correct dosage level,
(4) select a feeding frequency between five times per day and once per week,
(5) begin consumption of the medical food,
(6) periodically measure serum or salivary cortisol levels and adjust the medical food dosage,
(7) continue consumption until restful sleep is achieved or cortisol is lowered,
(8) consume the medical food on a maintenance basis as needed. This may be done at the treatment dosage or at a reduced dosage, and
(9) use a health care professional to supervise consumption of the sleep enhancement medical food.

For some patients, transfer factor, lactic acid generating bacteria, and glucans are taken together. For other patients, transfer factor, lactic acid generating bacteria, and glucans are taken at different times during the day or week. Component separation and consumption at different times are within the scope of this invention. Separate consumption and was recited in Ramaekers' U.S. Publication 20070128253, which benefits this application. The human body performs the mixing.

Other stress hormones may also be lowered by consuming transfer factor, lactic acid generating bacteria, and/or glucans. Hence, other stress hormones may be monitored.

The physiology between animals and humans is similar. But methods of treatment differ for humans from animals. Two differences involve (1) treatment durations and (2) feeding frequency. Humans typically have a wider range of feeding frequencies, and longer treatment durations than livestock. This reflects the underlying treatment guidelines.

For livestock, the guideline is economic. Feeding is operationally fixed. Changing the feeding frequency affects a rancher's profit.

For humans, the guideline is effectiveness. Cortisol levels increase in response to stressful thoughts, and subside with calming thoughts. Hence, longer treatment periods for humans are often used, relative to livestock. Several small sleep improvement food portions per day—rather than 1 large portion per day—are not difficult for humans to arrange. Consumption frequency may vary between five times per day and once per week.

Hence, method steps in this application are different from animal applications for which filing benefit is claimed. Human patients respond to the invented sleep improvement food in different time frames. Treatment periods vary.

Periodically measuring cortisol during treatment is recommended. Based on periodic cortisol measurement, dosages, frequency of feeding, and component proportions are optimized. Without measurement; the treatment program cannot be exact. With measurement, treatment can be better refined.

A 2003 study of people with chronic insomnia reported that cortisol levels were high only when their corresponding sleep was of poor quality. When they slept well, cortisol levels were lower. So, cortisol measurement is particularly important on sleepless nights.

Sleep disorders frequently overlap with other medical problems. And some of those other medical problems are also improved with a combination of transfer factor and lactic acid generating bacteria. Again, cortisol reduction and immune building are active.

An example is depression, providing that depression correlates with an excess of cortisol. With or without a sleep disorder, high cortisol depression responds well to a medical food created from transfer factor, lactic acid generating bacteria, and/or glucans.

In the United States, an estimated 6.7% of the U.S. population suffers from depression. This number corresponds to roughly 21 million people.

The Department of Psychiatry, University of California, San Francisco, Calif. performed studies linking cortisol to human depression. A total of seven studies comparing plasma or cortisol responses to psychological stressors in clinically depressed (MDD) and non-depressed (ND) individuals (N=196: 98 MDD, 98 ND; 83 men, 113 women; mean age=40 years) were included. Sample size-adjusted effects (Cohen's d statistic) were calculated and averaged across baseline (before stressor onset), stress (stressor onset up to 25 min after stressor offset), and recovery (more than 25 min after stressor offset) periods. Overall, MDD and ND individuals exhibited similar baseline and stress cortisol levels, but MDD patients had much higher cortisol levels during the recovery period than their ND counterparts. There was also a significant time of day effect. Afternoon studies were more likely to reveal higher baseline cortisol levels, blunted stress reactivity; and impaired recovery in MDD patients. This blunted reactivity-impaired recovery pattern observed among the afternoon studies was most pronounced in studies with older and more severely depressed patients.

The method of use for depression parallels that of sleep disorders, but different improvement metrics are appropriate. The medical food composition and dosage remain within the boundaries of this disclosure: (1) transfer factor and lactic acid generating bacteria, or (2) transfer factor, lactic acid generating bacteria, and glucans. Feeding dosages are based on weight, and feeding frequencies are between five times per day and once per week.

A second example of a health issue that frequently occurs in conjunction with sleep disorder is diabetes. Some categories of diabetes correlate with high cortisol.

Prolonged elevation of cortisol causes diabetic-level blood sugar. It does so by increasing insulin resistance in the body. Insulin is the hormone that causes the body to uptake sugar from the blood. The body then stores the sugar in various tissues, mainly the liver and in fat tissue. With elevated cortisol, your body doesn't respond as well to the insulin signal. Thus, there is less uptake of sugar from the blood, leaving more sugar in the blood. This high sugar level is seen in patients with Type II diabetes mellitus.

In the United States, 25.8 million children and adults—8.3% of the population—have diabetes. Deaths attributed to diabetic causes are roughly 69,000 per year.

The combination of transfer factor, lactic acid generating bacteria, and/or glucans has been shown to lower cortisol. The important effect is that the rate of blood sugar uptake is restored, and blood sugar levels return to a normal range.

Again, the method of treatment parallels the treatment of sleep disturbance. Both blood sugar levels and sleep quality improve simultaneously, leading to a dramatic change. The medical food composition remains within the boundaries of this disclosure: (1) transfer factor and lactic acid generating bacteria, or (2) transfer factor, lactic acid generating bacteria, and glucans. Feeding dosages are based on weight, and feeding frequencies are between five times per day and once per week.

Diabetes can be improved by the medical food, even if sleep disorder is not present.

A third example of a health problem that overlaps with sleep disorder is cardiovascular disease. Patients with the highest cortisol levels were five times as likely to die of heart attack, stroke or other cardiovascular causes over a period of six years. Cardiovascular disease (CVD) is the leading cause of death (roughly 600,000 per year) in the United States, and is responsible for 17% of national health expenditures. As the population ages, these costs are expected to increase substantially.

Researchers found that hair cortisol levels were a more important predictor of heart attack risk than established risk factors like high blood pressure and cholesterol.

Feeding the combination of transfer factor, lactic acid generating bacteria, and/or glucans improves sleeplessness and heart disease together or each health issue separately.

The method to improve cardiovascular patients with high cortisol parallels the method to improve sleep quality. The underlying cause of both is high cortisol. Metrics to follow the each improvement may differ, but the method of administering is similar. The medical food composition, dosage levels, and feeding frequencies remain within the boundaries of this sleep disorder disclosure. Feeding dosages are based on weight, and feeding frequencies are between five times per day and once per week.

A fourth example of a health issue that frequent occurs in conjunction with sleep disorder is Alzheimer's disease. In the early stages of Alzheimer's disease, cortisol reduction can slow the progression. The earlier cortisol reduction begins, the better the prognosis. The medical food based on transfer factor, lactic acid generating bacteria, and/or glucans helps sleep deprivation and early stage Alzheimer's because cortisol is reduced.

The U.S. incidence rate of Alzheimer's is 5.2 million, and growing as the population ages. It is a particularly cruel disease.

Over the long term, elevated cortisol kills brain cells (this is widely accepted). The worst part is that the hippocampus is the part of the brain most affected. The hippocampus serves two important roles. New memories are formed in the hippocampus, and the hippocampus limits the generation of more cortisol.

Dr. Dharma Khalsa describes how some older people lose 20-25% of the hippocampus cells. Hence, the hippocampus can no longer function as the cortisol feedback control circuit. Cortisol production builds, more hippocampus cells are lost, even more cortisol is produced, etc. A degenerative cascade develops. Dr. Robert Sapolsky at Stanford University confirmed Dr. Khalsa's scenario. Medical food feeding dosages are based on weight, and feeding frequencies are between five times per day and once per week.

A fifth example of insomnia co-existing with another health issue is breast cancer.

An estimated 232,340 new cases of invasive breast cancer and 39,620 breast cancer deaths were expected to occur among U.S. women in 2013. About eight in ten breast cases and nearly nine in ten breast cancer deaths will occur among women aged 50 years and older.

Current medical treatments for breast cancer are stressful in themselves. Increased cortisol levels are not unexpected. This is reflected in sleeplessness. Women with advanced breast cancer who have abnormal daytime levels of cortisol are significantly more likely to die sooner than patients with normal levels of the hormone.

The medical food based on transfer factor, lactic acid generating bacteria, and/or glucans helps both sleeplessness and breast cancer. As cortisol is reduced, sleep improves. And the improved immune system makes it easier to recover from chemotherapy, radiation, and surgery.

Feeding the combination of transfer factor, lactic acid generating bacteria, and/or glucans is useful, even in the absence of insomnia. But it serves a dual purpose in the presence of insomnia.

The medical food composition, dosage levels, and feeding frequencies remain within the boundaries of this sleep disorder disclosure. Feeding dosages are based on weight, and feeding frequencies are between five times per day and once per week.

The invention claimed is:

1. A food-based method for improving a sleep disorder for a human with high cortisol comprising:
combining transfer factor and lactic acid generating bacteria to create a medical food, where
said transfer factor includes polypeptides with a molecular weight below 10,000 Daltons;
feeding said medical food to said human, where
a dosage level is chosen based on said human's weight, and
the frequency of said feeding is between five times per day and once per week; and
continuing said feeding until restful sleep is achieved.

2. The food-based method of claim 1 wherein said high cortisol is defined as a cortisol level that exceeds the United States population average.

3. The food-based method of claim 1 wherein glucans are added to said medical food.

4. The food-based method of claim 3 wherein said glucans are derived from natural or hybrid mushrooms.

5. The food-based method of claim 1 further comprising:
measuring night time cortisol levels.

6. The food-based method of claim 5 further comprising:
adjusting said dosage levels based on said measuring.

7. The food-based method of claim 3 further comprising:
changing the relative proportions of transfer factor, lactic acid generating bacteria, and
glucans included within each said food dosage.

8. The food-based method of claim 1 wherein said transfer factor in each said dosage is present at 0.05 to 50 mg per pound of human body weight.

9. The food-based method of claim 1 wherein said lactic acid generating bacteria in each said dosage is present at 0.47 to 10 mg per pound of human body weight.

10. The food-based method of claim 9 wherein said lactic acid generating bacteria has a live count of 2.5 million colony forming units per ounce.

11. The food-based method of claim 3 wherein said glucans in each said dosage is present at 0.1 to 10 mg per pound of human body weight.

12. The food-based method of claim 3 wherein transfer factor transfer, lactic acid generating bacteria, and glucans are consumed at different times within a one week period.

13. An improvement upon U.S. Pat. No. 6,962,718, claim 6 (issued Nov. 8, 2005 to Joseph Ramaekers, a current inventor) which recites—
A formulation comprising pharmaceutically acceptable transfer factor and a pharmaceutically acceptable lactic acid generating bacteria wherein the amount of said transfer factor is from 10 mg to 10,000 mg per ounce of formulation;
wherein the improvement comprises the following method-of-use limitation,
feeding said formulation to sleep disorder patients to enhance restful sleep wherein said patients exhibit high cortisol levels.

14. The improvement claim in claim 13, wherein said feeding includes at least one step selected from a group including:
(a) optimizing proportions of transfer factor transfer and said lactic acid generating bacteria within said formulation to create a sleep improvement medical food, (b) choosing a dosage level of said formulation based on said patient's weight, (c) feeding said patient a dosage between five times per day and one time per week, (d) measuring said patient's cortisol levels periodically, (e) adjusting dosage levels based on periodic cortisol measurements, and (f) monitoring multiple stress hormones.

15. The improvement claim in claim 13, wherein glucans are added to said formulation.

16. The improvement claim in claim 15, wherein said glucans are present between at 0.1 to 10 mg per pound of patient's body weight.

17. A food-based method for improving sleep quality for a human when high cortisol levels are contributing to sleeplessness comprising:
combining transfer factor transfer and lactic acid generating bacteria to create a medical food, wherein
said transfer factor includes polypeptides with a molecular weight below 10,000 Daltons;
feeding said medical food to said human, wherein
a dosage level is chosen based on said human's weight, and
the frequency of said feeding is between five times per day and once per week; and
utilizing a health care professional or doctor to supervise said improving sleep quality.

18. A food-based method for improving depression for a human when high cortisol levels are present comprising:
combining transfer factor and lactic acid generating bacteria to create a medical food, wherein
said transfer factor includes polypeptides with a molecular weight below 10,000 Daltons;
feeding said medical food to said human, wherein
a dosage level is chosen based on said human's weight, and
the frequency of said feeding is between five times per day and once per week; and
continuing said feeding until symptoms of depression are reduced.

19. A food-based method for improving diabetes or high blood sugar for a human when high cortisol levels are present comprising:
combining transfer factor and lactic acid generating bacteria to create a medical food, wherein
said transfer factor includes polypeptides with a molecular weight below 10,000 Daltons;
feeding said medical food to said human, wherein
a dosage level is chosen based on said human's weight, and
the frequency of said feeding is between five times per day and once per week; and
continuing said feeding until blood sugar is reduced.

20. A food-based method for improving cardiovascular disease for a human when high cortisol levels are present comprising:
combining transfer factor and lactic acid generating bacteria to create a medical food, wherein
said transfer factor includes polypeptides with a molecular weight below 10,000 Daltons;
feeding said medical food to said human, wherein
a dosage level is chosen based on said human's weight, and
the frequency of said feeding is between five times per day and once per week; and
continuing said feeding until cortisol levels are reduced or until said cardiovascular disease improves.

21. A food-based method for slowing Alzheimer's progression for a human when high cortisol levels are present comprising:
combining transfer factor and lactic acid generating bacteria to create a medical food, wherein
said transfer factor includes polypeptides with a molecular weight below 10,000 Daltons;
feeding said medical food to said human, wherein
a dosage level is chosen based on said human's weight, and
the frequency of said feeding is between five times per day and once per week; and
continuing said feeding until reduced cortisol levels are achieved or said progression is slowed.

22. A food-based method for improving breast cancer treatment for a human when high cortisol levels are present comprising:
combining transfer factor and lactic acid generating bacteria to create a medical food, wherein
said transfer factor includes polypeptides with a molecular weight below 10,000 Daltons;
feeding said medical food to said human, where
a dosage level is chosen based on said human's weight, and
the frequency of said feeding is between five times per day and once per week; and
continuing said feeding during or after said breast cancer treatment.
